(19) United States
(12) Patent Application Publication
(10) Pub. No.: US 2012/0283660 A1
(43) Pub. Date: Nov. 8, 2012

(54) METHOD AND ASSEMBLY FOR A DRUG DELIVERY DEVICE

(75) Inventors: Christopher Jones, Tewkesbury (GB); Robert Veasey, Leamington Spa (GB); Garen Kouyoumjian, Leamington Spa (GB); Catherine Anne Macdonald, Ashby-de-la-Zouch (GB)

(73) Assignee: SANOFI-AVENTIS DEUTSCHLAND GMBH, Frankfurt am Main (DE)

(21) Appl. No.: 13/498,377

(22) PCT Filed: Sep. 29, 2010
(86) PCT No.: PCT/EP2010/064421

§ 371 Date: Jul. 24, 2012

(30) Foreign Application Priority Data
Sep. 30, 2009 (EP) 09171759.5

(51) Int. Cl. A61M 5/31; A61M 5/315

(52) U.S. Cl. 604/218; 604/187

(57) ABSTRACT

A method for securing a cartridge to a body for a drug delivery device is provided comprising the step of securing the cartridge to the body by means of a releasable connection to prevent accidental relative movement between the cartridge (4) and the body during assembly of the device. Afterwards, the releasable connection is modified irreversably into a non-releasable connection such that the cartridge is permanently secured to the body. Furthermore, an assembly for a drug delivery device is provided comprising a cartridge, a body, a first connection means and a second connection means. The first connection means and the second connection means are configured to mechanically cooperate to form a releasable connection. The releasable connection is suitable for releasably securing the cartridge to the body. The releasable connection prevents accidental relative movement between the cartridge and the body during assembly of the device. The first connection means and the second connection means are irreversably non-releasably secured to each other.

### CROSS REFERENCE TO RELATED APPLICATIONS

The present application is a U.S. National Phase Application pursuant to 35 U.S.C. §371 of International Application No. PCT/EP2010/064421 filed Sep. 29, 2010, which claims priority to European Patent Application No. 09171759.5 filed on Sep. 30, 2009. The entire disclosure contents of these applications are herewith incorporated by reference into the present application.

### FIELD OF INVENTION

This disclosure relates to a method for securing a cartridge to a body for a drug delivery device. The disclosure further relates to an assembly for a drug delivery device.

### BACKGROUND

In a drug delivery device a piston within a cartridge containing a plurality of doses of a drug may often be displaced with respect to the cartridge in a distal direction by a drive member. Thereby, a dose of the drug may be expelled from the cartridge.

Drug delivery devices are described in documents EP 1 923 083 A1 and WO 2006/063472 A1.

It is an object of the present disclosure to facilitate provision of an improved drug delivery device, for example a device with high dose accuracy. Furthermore, an assembly for an improved drug delivery device is provided.

This object may be achieved by the subject matter of the independent claims. Further features are the subject matter of the dependent claims.

### SUMMARY

According to one aspect a method for securing a cartridge to a body for a drug delivery device is provided. The cartridge may be secured to the body, in particular with respect to the body, by means of a releasable connection. The releasable connection may prevent relative axial movement, in particular between the cartridge and the body, during assembly of the device. Afterwards, the releasable connection may be modified irreversably into a non-releasable connection such that the cartridge is permanently secured to the body. In particular, after the connection is made non-releasable the cartridge may not be unsecured from the body without destroying the non-releasable connection.

Another aspect relates to an assembly for a drug delivery device. The assembly comprises a cartridge. The assembly comprises a body. The assembly may comprise a first connection means. The assembly may comprise a second connection means. The first connection means and the second connection means may be configured to mechanically cooperate to form a releasable connection. The releasable connection may be suitable for releasably securing the cartridge to the body. The releasable connection may be adapted to prevent relative axial movement, in particular between the cartridge and the body, preferably during assembly of the device. The first connection means and the second connection means may be irreversably non-releasably secured to each other.

The releasable connection may keep the first connection means and the second connection means secured to each other. After checking whether other components of the drug delivery device have been assembled properly the first connection means and the second connection means may be non-releasably secured to each other by modifying the releasable connection.

The drug delivery device may be an injection device. The drug delivery device may be a pen-type device, e.g. a pen-type injector. The cartridge may hold a plurality of doses of a drug. Preferably, the drug comprises a liquid medication, such as long-acting or short-acting insulin, heparin or growth hormones. The drug delivery device may be designed such that it may accommodate cartridges of different sizes. Additionally or alternatively, the drug delivery device may be designed such that it may accommodate cartridges of different shapes.

The releasable connection may be modified into a non-releasable connection for permanently securing the cartridge to the body. Preferably, the cartridge is irreversably secured to the body such that the cartridge is prevented from moving axially with respect to the body. Additionally or alternatively, the cartridge may be permanently secured against rotational movement with respect to the body.

According to an embodiment, a drive member is retained in the body. A piston may be retained in the cartridge. The cartridge and the drive member may be adjusted with respect to one another such that the piston abuts the drive member before the releasable connection is modified.

According to an embodiment, the cartridge is securable to the body by means of the releasable connection with different securing-positions.

In an initial state of the drug delivery device there may be a gap between the drive member and the piston. The gap may arise from manufacturing and/or assembly tolerances of components, e.g. drive member and piston, of the drug delivery device. The size of the gap may vary. However, in the delivery condition, i.e. when delivering a set dose of the drug, a gap between the drive member and the piston may affect the dose accuracy, because the drive member has to close the gap before the piston may be advanced and the drug may be expelled. Because the cartridge may be secured to the body in different axial positions, a reduction of differently sized gaps between the drive member and the piston may be enabled and hence, good dose accuracy may be guaranteed.

Preferably, the drive member is configured to displace the piston axially with respect to the cartridge for expelling a dose of the drug, in particular when the drug delivery device is operated. The drive member may be a piston rod. Preferably, the releasable connection is modified when the cartridge is in an axial position with respect to the body in which all play between the drive member and the piston and other components which are operated for dose delivery is taken up. User-operated steps for reducing play between the drive member and the piston may thus be redundant.

According to an embodiment, the releasable connection achievable by cooperation of the first connection means and the second connection means is modified. The modified releasable connection may be provided for irreleasably securing the cartridge to the body.

Modifying the releasable connection may comprise deformation of at least a portion of at least one of the connection means, for example by applying mechanical force to at least a portion of at least one of the connection means. The modified releasable connection may, for instance, comprise a permanently locked, e.g. deformed, screw thread. Additionally or alternatively, modifying the releasable connection may comprise applying an adhesive to at least a portion of at least one of the connection means and securing the first connection means and the second connection means to each other by means of the adhesive. Additionally or alternatively, modifying the releasable connection may comprise welding of at least a portion of the connection means such that the first connection means and the second connection means may be non-releasably joined by a weld.

According to one aspect a method for assembling a drug delivery device is provided. The method may comprise the method of permanently securing the cartridge to the body of the drug delivery device as described above. Modification of the releasable connection may be performed while assembling the drug delivery device.

In this way, a very user-friendly drug delivery device may be provided as user-operated steps for making the drug delivery device ready for a first operation may be redundant. In addition, the drug delivery device may provide a high dose accuracy because play between the drive member and the piston may have been reduced or even completely removed during assembly of the drug delivery device. Underdosing of the drug resulting from manufacturing tolerances, which may have dangerous consequences for the user, may be prevented in this way.

According to an embodiment, the releasable connection is an adjustable connection. The cartridge and the body may be irreversably secured to each other by means of a material-locking connection.

The cartridge may be permanently secured to the body by material engagement of the first connection means and the second connection means. The material-locking connection may for example comprise a portion of the first connection means and the second connection means in which both connection means are joined by a weld. Additionally or alternatively, the material-locking connection may comprise use of a separate connecting material such as an adhesive. The separate connecting material may be applied to at least a portion of at least one of the connection means such that upon interaction the first connection means and the second connection means may be permanently secured to each other by means of the separate connecting material.

According to an embodiment, a cartridge holder is provided. The cartridge may be retained in the cartridge holder. The cartridge holder may be irreversably secured to the body by means of the first connection means and the second connection means. Thus, the cartridge may be secured to the body via the cartridge holder. Alternatively, the cartridge may be secured directly to the body. In this case the cartridge holder may be redundant.

Preferably, the cartridge is secured in the cartridge holder. The releasable connection formed by mechanical cooperation of the first connection means and the second connection means may be modified. Due to said modification the cartridge holder may be irreversibly non-releasably secured to the body. In this way, the cartridge may be permanently secured axially and/or rotationally within the cartridge holder against displacement with respect to the body.

Of course, features relating to different aspects described above may be combined with each other.

Further features and refinements become apparent from the following description of the exemplary embodiments in connection with the accompanying figures.

### BRIEF DESCRIPTION OF THE FIGURES

FIG. 1 schematically shows a sectional side view of an exemplary drug delivery device,

FIG. 2 schematically shows a sectional side view of a part of the drug delivery device of FIG. 1,

FIG. 3 schematically shows a sectional side view of a part of the drug delivery device of FIG. 1 in an unassembled condition,

FIG. 4 schematically shows a sectional side view of a part of the drug delivery device of FIG. 1 in an assembled condition.

Like elements, elements of the same kind and identically acting elements may be provided with the same reference numerals in the figures.

### DETAILED DESCRIPTION

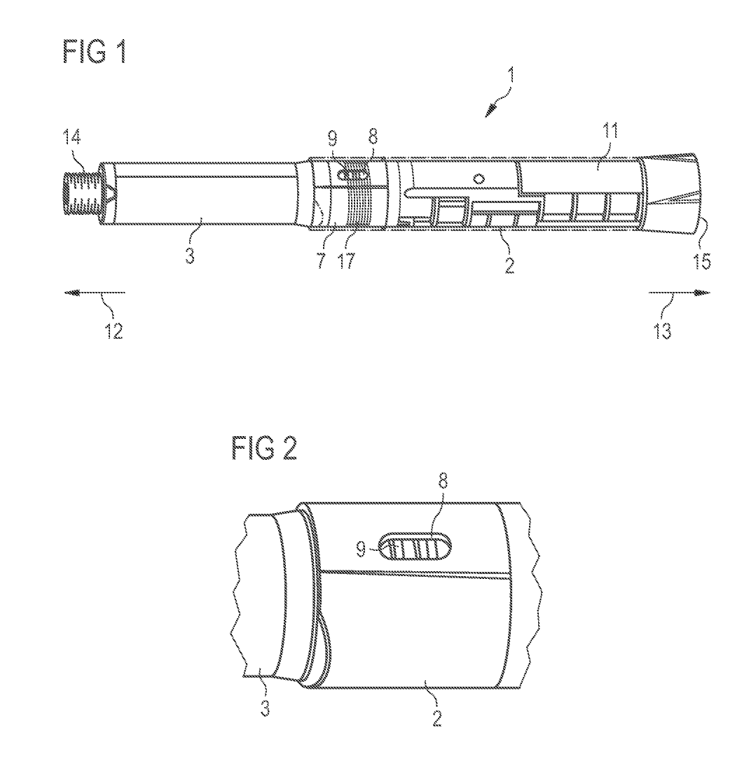

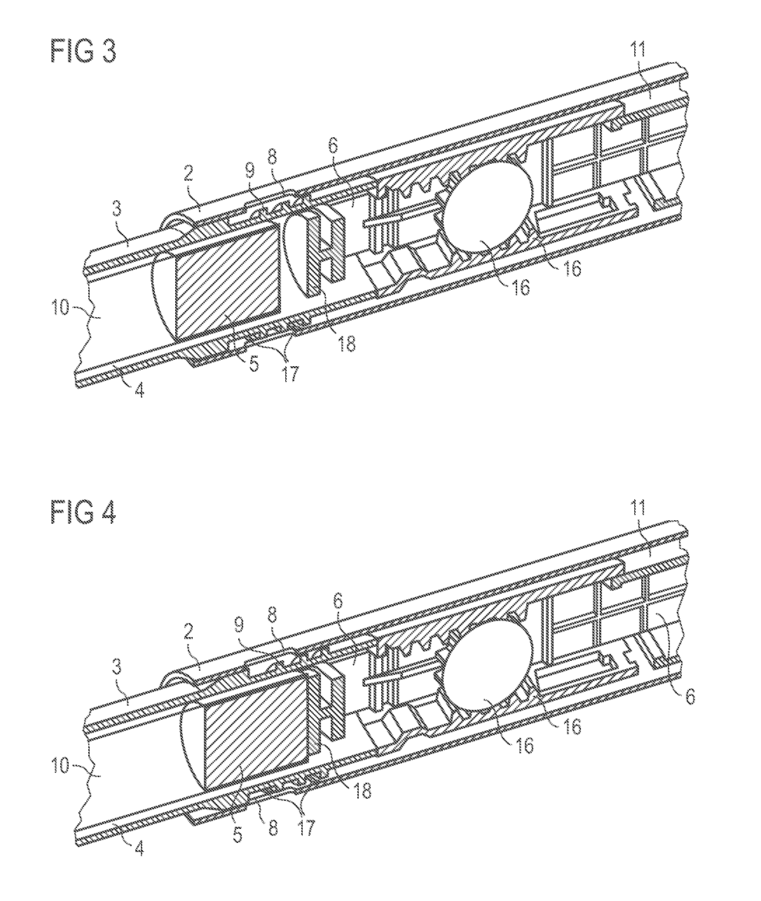

In FIG. 1 a drug delivery device 1 is shown. The drug delivery device comprises a body 2. The drug delivery device 1 comprises a cartridge 4, which is indicated in FIGS. 3 and 4. The cartridge 4 is retained within a cartridge holder 3. The cartridge holder 3 may secure the cartridge 4 mechanically. The cartridge 4 contains a drug 10 (see FIGS. 3 and 4), preferably a plurality of doses of the drug 10. The drug 10 preferably comprises a liquid medication, for example comprising insulin, like short-acting or long-acting insulin, heparin or growth hormones.

The term “drug”, as used herein, preferably means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:
H-(Lys)4-des Pro36, des Pro37 Exendin-4(1-39)-NH2,
H-(Lys)5-des Pro36, des Pro37 Exendin-4(1-39)-NH2,
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39); or
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39),
wherein the group -Lys6-NH2 may be bound to the C-terminus of the Exendin-4 derivative;
   or an Exendin-4 derivative of the sequence
H-(Lys)6-des Pro36 [Asp28] Exendin-4(1-39)-Lys6-NH2,
des Asp28 Pro36, Pro37, Pro38Exendin-4(1-39)-NH2,
H-(Lys)6-des Pro36, Pro38 [Asp28] Exendin-4(1-39)-NH2,
H-Asn-(Glu)5des Pro36, Pro37, Pro38 [Asp28] Exendin-4(1-39)-NH2,
des Pro36, Pro37, Pro38 [Asp28] Exendin-4(1-39)-(Lys)6-NH2,
H-(Lys)6-des Pro36, Pro37, Pro38 [Asp28] Exendin-4(1-39)-(Lys)6-NH2,
H-Asn-(Glu)5-des Pro36, Pro37, Pro38 [Asp28] Exendin-4(1-39)-(Lys)6-NH2,
H-(Lys)6-des Pro36 [Trp(O2)25, Asp28] Exendin-4(1-39)-Lys6-NH2,
H-des Asp28 Pro36, Pro37, Pro38 [Trp(O2)25] Exendin-4(1-39)-NH2,
H-(Lys)6-des Pro36, Pro37, Pro38 [Trp(O2)25, Asp28] Exendin-4(1-39)-NH2,
H-Asn-(Glu)5-des Pro36, Pro37, Pro38 [Trp(O2)25, Asp28] Exendin-4(1-39)-NH2,
des Pro36, Pro37, Pro38 [Trp(O2)25, Asp28] Exendin-4(1-39)-(Lys)6-NH2,
H-(Lys)6-des Pro36, Pro37, Pro38 [Trp(O2)25, Asp28] Exendin-4(1-39)-(Lys)6-NH2,
H-Asn-(Glu)5-des Pro36, Pro37, Pro38 [Trp(O2)25, Asp28] Exendin-4(1-39)-(Lys)6-NH2,
H-(Lys)6-des Pro36 [Met(O)14, Asp28] Exendin-4(1-39)-Lys6-NH2,
des Met(O)14 Asp28 Pro36, Pro37, Pro38 Exendin-4(1-39)-NH2,
H-(Lys)6-desPro36, Pro37, Pro38 [Met(O)14, Asp28] Exendin-4(1-39)-NH2,
H-Asn-(Glu)5-des Pro36, Pro37, Pro38 [Met(O)14, Asp28] Exendin-4(1-39)-NH2,
des Pro36, Pro37, Pro38 [Met(O)14, Asp28] Exendin-4(1-39)-(Lys)6-NH2,
H-(Lys)6-des Pro36, Pro37, Pro38 [Met(O)14, Asp28] Exendin-4(1-39)-(Lys)6-NH2,
H-Asn-(Glu)5 des Pro36, Pro37, Pro38 [Met(O)14, Asp28] Exendin-4(1-39)-(Lys)6-NH2,
H-Lys6-des Pro36 [Met(O)14, Trp(O2)25, Asp28] Exendin-4(1-39)-Lys6-NH2,
H-des Asp28 Pro36, Pro37, Pro38 [Met(O)14, Trp(O2)25] Exendin-4(1-39)-NH2,
H-(Lys)6-des Pro36, Pro37, Pro38 [Met(O)14, Asp28] Exendin-4(1-39)-NH2,
H-Asn-(Glu)5-des Pro36, Pro37, Pro38 [Met(O)14, Trp(O2)25, Asp28] Exendin-4(1-39)-NH2,
des Pro36, Pro37, Pro38 [Met(O)14, Trp(O2)25, Asp28] Exendin-4(1-39)-(Lys)6-NH2,
H-(Lys)6-des Pro36, Pro37, Pro38 [Met(O)14, Trp(O2)25, Asp28] Exendin-4(S1-39)-(Lys)6-NH2,
H-Asn-(Glu)5-des Pro36, Pro37, Pro38 [Met(O)14, Trp(O2)25, Asp28] Exendin-4(1-39)-(Lys)6-NH2;
   or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

The cartridge 4 may comprise an outlet (not explicitly shown). The outlet may be covered by a membrane (not explicitly shown). The drug 10 can be dispensed from the cartridge 4 through the outlet when the membrane is pierced. The drug delivery device 1 comprises engaging means 14, preferably for securing a needle assembly (not explicitly shown) to the cartridge holder 3. The needle assembly may pierce the membrane, when the drug delivery device 1 is operated.

The drug delivery device 1 comprises a drive member (see drive member 6 in FIGS. 3 and 4) which is explained later on in more detail. The device 1 comprises a piston 5 (not explicitly shown in FIG. 1, see FIGS. 3 and 4). The drug delivery device 1 comprises a dose member 11. The drug delivery device 1 comprises a dose button 15. The dose member 11 and the dose button 15 may be movable with respect to the body 2 for setting and for delivering a dose of the drug 10 from the cartridge 4.

The drug delivery device 1 and the body 2 have a distal end and a proximal end. The distal end of the device 1 is indicated by arrow 12, which refers to that end of the drug delivery device 1 which is closest to a dispensing end of the drug delivery device 1. The proximal end of the device 1 is indicated by arrow 13 referring to that end of the device 1 which is furthest away from the dispensing end of the device 1.

The drug delivery device 1 may be a pen-type device, in particular a pen-type injector. The device 1 may be a disposable device. The device may be configured to dispense fixed doses of the drug 10 or variable, preferably user-settable doses of the drug 10. It may be crucial that there is no gap between the drive member 6 and the piston 5 in the delivery condition as a gap may reduce dose accuracy, because the drive member 6 has to close the gap before the piston 5 may be advanced and the drug 10 may be expelled. In an assembled condition of the device 1, the gap may arise from manufacturing and/or assembly tolerances of the components of the device 1, e.g. of the drive member 6 and piston 5. The drug delivery device 1 may be a manually, in particular a non-electrically, driven device.

The body 2 may be designed to enable a safe and comfortable handling of the drug delivery device 1. The body 2 may be configured to house, fix, protect and guide inner components of the drug delivery device 1, e.g. drive member 6, dose member 11. Preferably, the body 2 limits or prevents the exposure of the inner components to contaminants such as liquid, dirt or dust. The body 2 may be a unitary or a multipart component. The body 2 may comprise a tubular or a cylindrical shape, as shown in FIG. 1. Alternatively, the body 2 may comprise a non-tubular shape.

The drive member 6 may be a piston rod, for example. The drive member 6 has a distal end and a proximal end. The distal end of the drive member 6 may be the end which is closest to the distal end 12 of the drug delivery device 1 when the drive member 6 has been assembled in the device 1. The proximal end of the drive member 6 may be the end which is furthest away from the distal end 12 of the drug delivery device 1 when the drive member 6 has been assembled in the device 1.

The drive member 6 may operate through the body 2 of the drug delivery device 1. The drive member 6 may be designed to transfer axial movement through the drug delivery device 1, for example for the purpose of delivering the drug 10.

The drive member 6 may be made of a flexible or a rigid material. The drive member 6 may have a circular or a non-circular cross-section. The drive member 6 may be of unitary or multipart construction. A bearing member 18 (not explicitly shown in FIG. 1, see FIGS. 3 and 4) may be located at the distal end of the drive member 6. The bearing member 18 may abut the piston 5, facilitating interaction of the piston 5 and the drive member 6.

The piston 5 may be slideably retained within the cartridge 4 of the drug delivery device 1. The piston 5 is movable with respect to the cartridge 4. The piston 5 may seal the cartridge 4 proximally. Movement of the piston 5 in the distal direction with respect to the cartridge 4 causes the drug 10 to be dispensed from the cartridge 4 through the outlet.

The drug delivery device 1 may comprise a drive mechanism 16 (not explicitly shown in FIG. 1, see FIGS. 3 and 4). The drive mechanism 16 may be retained within the body 2. When delivering a dose of the drug 10, the drive member 6 may be displaced in the distal direction with respect to the body 2 due to operation of the drive mechanism 16. The dose member 11 may be part of the drive mechanism 16. The user may displace the dose member 11 in the proximal direction with respect to the body 2 for setting a dose of the drug 10. Afterwards, the user may displace the dose member 11 in the distal direction with respect to the body 2 for delivering the set dose of the drug 10. The dose button 15 may be integrally formed with the dose member 11 or may be connected to the dose member 11. The dose button 15 may be secured against rotational movement with respect to the dose member 11. The user may push the dose button 15 in the proximal direction for delivering the set dose of the drug 10.

The cartridge holder 3 and the body 2 may be adapted to releasably engage with each other. The cartridge holder 3 may be connectable to the body 2 of the drug delivery device 1, preferably by means of a releasable connection 7. The cartridge holder 3 may comprise a first connection means 9. The first connection means 9 may be arranged in the proximal end section of the cartridge holder 3. The first connection means 9 may comprise a thread, for example. Preferably, the first connection means 9 comprises an outer thread of the cartridge holder 3. The body 2 may comprise at least one aperture 8. The at least one aperture 8 may be arranged in the distal end section of the body 2. The first connection means 9 may be accessible and visible through the aperture 8 when the cartridge holder 3 is connected to the body 2.

The body 2 may comprise a second connection means 17. The second connection means 17 may comprise an inner or an outer thread, for example. Preferably, the second connection means 17 comprises an inner thread of the body 2. The second connection means 17 may be arranged in the distal end section of the body 2. In particular, the second connection means 17 may be arranged at the end of the body 2 facing to the cartridge holder 3.

The first connection means 9 and the second connection means 17 may be configured to mechanically cooperate to form the releasable connection 7. The releasable connection 7 may be an adjustable connection. Non-adjustable connections are snap-fit or pinned connections, for example. Adjustable connections are screwed, clamped or threaded connections, for example. The releasable connection 7 may be a threaded connection. The releasable connection 7 may be suitable for releasably securing the cartridge holder 3 and, hence, the cartridge 4, to the body 2 of the drug delivery device 1.

The cartridge holder 3 and the cartridge 4 may be securable to the body 2 by means of the releasable connection 7 in a plurality of different axial securing-positions. The different securing-positions may allow a gap of variable width which is in an initial assembly state of the drug delivery device 1 between the piston 5 and the drive member 6 and between members of the drive mechanism 16 to be accommodated. The gap may be minimized or eliminated during assembly while securing the cartridge holder 3 and the cartridge 4 to the body 2. In this way, a priming step which may be necessary for ordinary drug delivery devices 1 for closing the gap between the drive member 6 and the piston 5 and, hence, for guaranteeing an accurate first dose from the drug delivery device, may be redundant. Preferably, the cartridge holder 3 and, hence, the cartridge 4 is axially secured to the body 2 by means of the releasable connection 7 in a securing-position such that the piston 5 abuts the drive member 6. Accordingly, the cartridge holder 3 may be axially secured to the body 2 in a securing-position such that other members of the drive mechanism 16 abut each other.

Additionally, the first connection means 9 and the second connection means 17 may serve for irreversably non-releasably securing the cartridge holder 3 and, hence, the cartridge 4 to the body 2. For this purpose, the first connection means 9 and the second connection means 17 may be irreversably non-releasably secured to each other. In this case, the first connection means 9 and the second connection means 17 may mechanically cooperate to form a modified connection 7. The releasable connection 7 may be modified into a non-releasable connection 7 for permanently securing the cartridge holder 3 and the cartridge 4 to the body 2.

Modification of the releasable connection 7 may comprise deformation of at least a portion of the first connection means 9 or a portion of the second connection means 17 or deformation of at least a portion of both connection means, for example. The modified releasable connection 7 may comprise a permanently locked screw thread, for example. Additionally or alternatively, modification of the releasable connection 7 may comprise applying an adhesive to at least a portion of the first connection means 9 or a portion of the second connection means 17 or to at least a portion of both connection means, for example. Additionally or alternatively, modification of the releasable connection 7 may comprise welding or heat staking of the first connection means 9 and the second connection means 17.

After having modified the releasable connection 7, the cartridge holder 3 and, hence, the cartridge 4, and the body 2 may be permanently secured to each other by a material-locking connection. The material-locking connection may comprise, for example, a portion of the first connection means 9 and the second connection means 17 in which both connection means are joined by deformed material, a weld or an adhesive keeping the first connection means 9 and the second connection means 17 permanently secured to each other.

The cartridge 4 may be clamped between the cartridge holder 3 and the drive member 6. In particular at a distal side the cartridge 4 may interact with the cartridge holder 3 and at a proximal side the cartridge 4 may interact with the drive member 6. Hence, due to non-releasably securing the cartridge holder 3 to the body 2 the cartridge 4 may be permanently secured to the body 2 such that the cartridge 4 may be permanently secured against axial movement with respect to the body 2. Additionally, the cartridge 4 may be secured against rotational movement with respect to the body 2.

The cartridge holder 3 and the cartridge 4 may be permanently secured to the body 2 in the axial securing-position with the drive member 6 abutting the piston 5. Thus, due to the non-releasable connection 7, the piston 5 and the drive member 6 may be permanently held in abutment. Accordingly, members of the drive mechanism 16 may be permanently held in abutment. Hence, in the supplied condition from the manufacturer there is no gap between the piston 5 and the drive member 6 and/or between members of the drive mechanism 16. Accordingly, a user-operated priming step for closing the gap between the drive member 6 and the piston 5 and/or between the other members of the drive mechanism 16 during the first actuation of the drug delivery device 1 for setting and/or delivering a dose may be redundant. This improves the safety of the drug delivery device because users sometimes forget to undertake a priming step and therefore administer an incorrect first dose. Small discrepancies of the dose dispensed from a desired amount may have dangerous consequences for the user.

The drug delivery device 1 may for example be configured for setting and delivering doses of 30 U or greater, for example a dose of 50 U or greater. Alternatively, the drug delivery device 1 may provide for doses of 5 U or less or any dose in-between while having good dose accuracy.

FIG. 2 schematically shows a sectional side view of a part of the drug delivery device of FIG. 1. In particular, FIG. 2 shows the distal end section of the body 2 and the proximal end section of the cartridge holder 3. The cartridge holder 3 and the body 2 may be releasably secured to each other by means of the releasable connection 7. The releasable connection 7, in particular the first connection means 9, may be accessible and visible through the aperture 8.

FIG. 3 schematically shows a sectional side view of a part of the drug delivery device of FIG. 1 in an unassembled condition.

FIG. 4 schematically shows a sectional side view of a part of the drug delivery device of FIG. 1 in an assembled condition.

In particular, FIG. 3 shows the drug delivery device 1 with a considerable gap between bearing member 18 of the drive member 6 and the piston 5. The cartridge holder 3 may thus be still releasably secured to the body 2 by means of the releasable connection 7. Accordingly, the cartridge holder 3 and thus, the cartridge 4, may not yet be positioned in the final axial securing-position of the cartridge 4 with respect to the body 2. The cartridge holder 3, and thus the cartridge 4 may still be axially displaced relative to the drive member 6. The releasable connection 7 prevents accidental relative movement between the cartridge 4 and the body 2 during the assembly process. In particular, the releasable connection 7 may prevent relative axial movement between the cartridge 4 and the body 2 during the assembly process. Hence, a stable relative axial position of the cartridge 4 and the body 2 is established due to the releasable connection 7. In particular, relative axial movement arising, for example, due to an axial force applied on the cartridge 4 and/or the body 2 during assembly may be prevented by means of the releasable connection 7 as the releasable connection 7 may counteract said force to prevent the relative axial movement. If the releasable connection 7 comprises a thread, the thread is preferably self-locking

FIG. 4 shows the drug delivery device 1 after the gap between the piston 5 and the bearing member 18 of drive member 6 has been minimized. The cartridge holder 3 and thus, the cartridge 4, may be positioned at the final axial securing-position with respect to the body 2. Accordingly, the releasable connection 7 may be modified for irreversably non-releasably securing the cartridge holder 3 to the body 2.

Preferably, modification of the releasable connection 7 may be performed while assembling the drug delivery device 1. In this way, the drug delivery device 1 may be ready for operation when supplied to the user. Hence, no further user-operated steps, such as a priming step, may be required for making the drug delivery device 1 ready for operation, e.g. for minimizing the distance between the piston 5 and the drive member 6 and/or between other members of the drive mechanism 16. In this way, a particularly user-friendly, easily handled and safe drug delivery device 1 may be provided.

In the following, assembly of the drug delivery device 1 is described:

The cartridge holder 3, the cartridge 4 and the body 2 as described in connection with the description of FIGS. 1 and 2 may be provided for. The cartridge 4 may be retained in the cartridge holder 3 (see FIG. 3).

The position of the cartridge holder 3 and hence, the cartridge 4, may be adjusted with respect to the body 2, in particular with respect to the drive member 6 by releasably securing the cartridge holder 3 to the body 2 by means of the releasable connection 7 and moving the cartridge holder 3 proximally with respect to the body 2 in the different securing-positions (see FIG. 3).

When the piston 5 abuts the drive member 6 there is no or reduced play between members of the drive mechanism 16. In other words, there is no allowance for relative movement between the members of the drive mechanism 16. When there is no play between the piston 5 and the drive member 6 the cartridge holder 3 and thus, the cartridge 4, is in the final axial securing-position (see FIG. 4).

A further step may be performed to detect whether the piston 5 abuts the drive member 6. Said step may comprise detecting the force or torque necessary to displace the cartridge holder 3 proximally with respect to the body 2. Contact between the piston 5 and the drive member 6 may be detected for example by determining whether the force or torque exceeds a predetermined value. Additionally or alternatively, said step may comprise measurements of the position of the piston 5 and the position of the drive member 6 in order to calculate the axial position of the piston 5 required for abutment of the piston 5 and the drive member 6.

After having moved the piston 5 and the drive member 6 into abutment the releasable connection 7 may be modified for permanently irreleasably securing the cartridge holder 3 to the body 2 such that the cartridge holder 3 and the cartridge 4 may be permanently prevented from axial displacement with respect to the body 2 (see FIG. 4). This modification is facilitated by aperture 8, which allows the first connection means 7 to be accessed from the outside, for example for applying adhesive or for irradiation with a laser for a laser-weld or heat staking Alternatively, laser-weld, adhesive or heat staking may be applied without having an aperture 8.

After modification of the releasable connection 7 the drive member 6 permanently abuts the piston 5 and the play between members of the drive mechanism 16 is permanently diminished for guaranteeing good dose accuracy.

As described above the cartridge 4 may be secured to the body 2 via the cartridge holder 3. Alternatively, the cartridge 4 may be directly secured to the body 2. In this case the cartridge holder 3 may be redundant. Permanently directly securing the cartridge 4 to the body 2 may be performed in the same way as described above for the cartridge holder 3.

The steps of adjusting the cartridge holder 3 to the body 2 and irreversably permanently modifying the releasable connection 7 may be independent of the drive mechanism 16 of the drug delivery device 1. No further components for the drug delivery device 1 are required. Consequently, the drug delivery device 1 may comprise a small number of components, hence being less prone to errors.

As the releasable connection 7 may be easily modified for irreversably non-releasably securing the cartridge holder 3 and the cartridge 4 to the body 2 the method may be especially cost effective and hence, especially suitable for assembling disposable drug delivery devices 1.

The above described method may be especially suitable for assembling fixed dose drug delivery devices 1. Alternatively, the method may be applicable for assembling variable dose drug delivery devices 1.

Other implementations are within the scope of the following claims. Elements of different implementations may be combined to form implementations not specifically described herein.

1. A method for securing a cartridge to a body for a drug delivery device, comprising the following steps:
securing the cartridge to the body by means of a releasable connection, to prevent accidental relative movement between the cartridge and the body during assembly of the device,
modifying the releasable connection irreversably into a non-releasable connection such that the cartridge is permanently secured to the body.

2. The method of claim 1, wherein the cartridge is securable to the body by means of the releasable connection in different securing-positions.

3. The method of claim 1, wherein a drive member is retained in the body and a piston is retained in the cartridge, and wherein the cartridge and the drive member are adjusted with respect to one another such that the piston abuts the drive member before the releasable connection is modified.

4. The method according to claim 3, comprising the step of detecting whether the piston abuts the drive member and modifying the releasable connection after abutment of the piston and the drive member.

5. The method according to claim 1, wherein the releasable connection comprises a connection means and, wherein modifying the releasable connection comprises deformation of at least a portion of the connection means.

6. The method according to claim 1, wherein modifying the releasable connection comprises applying an adhesive to at least a portion of the connection means.

7. A method for assembling a drug delivery device comprising the method of permanently securing the cartridge to the body for the drug delivery device as claimed in claim 1, wherein the modification of the releasable connection is performed while assembling the drug delivery device.

8. An assembly for a drug delivery device, comprising a cartridge, a body, a first connection means and a second connection means, wherein the first connection means and the second connection means are configured to mechanically cooperate to form a releasable connection which is suitable for releasably securing the cartridge to the body to prevent accidental relative movement between the cartridge and the body during assembly of the device,
characterized in that the first connection means and the second connection means are irreversably non-releasably secured to each other.

9. The assembly of claim 8, wherein the first connection means and the second connection means irreleasably secure the cartridge to the body.

10. The assembly of claim 9, wherein the releasable connection achievable by cooperation of the first connection means and the second connection means is a modified releasable connection for irreleasably securing the cartridge to the body.

11. The assembly according to claim 10, wherein the releasable connection is an adjustable connection, and wherein the cartridge and the body are irreversably secured to each other by means of a material-locking connection.

12. The assembly according to claim 9, wherein the cartridge is irreversably secured to the body such that the cartridge is irreversably secured against axial movement with respect to the body.

13. The assembly according to claim 8, wherein a drive member is retained in the body and a piston is retained in the cartridge, and wherein the drive member is configured to displace the piston axially with respect to the cartridge, and wherein the drive member abuts the piston.

14. The assembly according to claim 8, wherein the modified releasable connection comprises a permanently locked screw thread.

15. The assembly according to claim 8, comprising a cartridge holder, wherein the cartridge is retained in the cartridge holder and the cartridge holder is irreversably secured to the body by means of the first connection means and the second connection means.
